(19) United States
(12) Patent Application Publication
(10) Pub. No.: US 2012/0070911 A1
(43) Pub. Date: Mar. 22, 2012

(54) Method of Detecting and Quantifying Analytes of Interest in a Liquid and Implementation Device

(75) Inventors: Jean-Pierre Peyrade, Ramonville Saint Agne (FR); David Peyrade, Grenoble (FR); Christophe Vieu, Auzeville Tolosane (FR); Laurent Malaquin, Linas (FR)

(73) Assignees: INSTITUT CURIE, Paris Cedex (FR); CENTRE NATIONAL DE LA RECHERCHE SCIENTIFIQUE, Paris (FR)

(21) Appl. No.: 13/258,580

(22) PCT Filed: Mar. 29, 2010
(86) PCT No.: PCT/FR10/00263

§ 371 Date: Dec. 6, 2011

(30) Foreign Application Priority Data
Mar. 31, 2009 (FR) 09 01573

(51) Int. Cl. G01N 33/53; G01N 21/78; G01N 21/64; B82Y 15/00

(52) U.S. Cl. 436/501; 422/69; 977/778; 977/902

(57) ABSTRACT

The invention provides a method of detecting and quantifying analytes present in a solution, which is portable, rapid, inexpensive, selective and ultra-sensitive. For this purpose, the subject of the invention is a method of detecting and quantifying analytes of interest (2) in a specimen (1) of liquid obtained from a mother liquor, the liquid being able to evaporate in an atmosphere (Atm) under defined evaporation conditions, the method comprising the following steps: b) the specimen (1) is deposited on a substrate (10) having a microstructured or nanostructured surface (20) defining analyte capture probes, in order for the liquid specimen to at least partially cover the structured surface of the substrate; c) the specimen undergoes controlled evaporation (5) in the vicinity (VT) of a liquid/substrate/atmosphere triple line (T), in such a way that this triple line moves, at a controlled rate, over the structured surface of the substrate as the liquid evaporates into the atmosphere, and so that the target analytes are captured, by convective assembly and directed capillary action, by the probes; and d) the structured surface of the substrate obtained after step c) is analysed.

The invention relates to a method of detecting and quantifying analytes of interest in a liquid and to an implementation device.

The required analytes of interest will be called “targets” hereinafter.

The method according to the invention makes it possible to reach limits of resolution of up to five nano-objects per milliliter, or less than an attomolar concentration of molecules. The liquid can be complex, i.e. it can comprise several types of analytes, only a proportion of which is of interest for detection.

It has been shown in numerous works that the increasing use of pesticides (fungicides, insecticides, herbicides), raticides, molluscicides, fertilizers, detergents, hormones and medicinal products, disperses the molecules that are included in their composition in the environment and therefore in plants and foodstuffs.

Moreover, the reactions of combustion of fuels discharge numerous nanoparticles, lead and graphite into the atmosphere.

The scheduled applications of functional nanoparticles also pose the problem of their dispersal in the natural environment.

To evaluate the risks of these molecules and nanoparticles to the environment and to humans it is necessary to detect them, to understand their mobility, their reactivity, their ecotoxicity and their persistence. The important parameters required are in particular detection of their presence and monitoring of the evolution of said presence over time.

In fact, to be able to detect and analyze the analytes present in water, air, soil, plants or foodstuffs is an important environmental challenge.

It is also a medical challenge. To be able to quickly obtain the composition of human specimens such as serum, blood or organs, to capture DNA strands, proteins and circulating biomarkers (circulating biomarkers are the precursors of metastatic recurrences of cancers), to capture bacteria or viruses, detect spores, at a very low level of concentration, is essential for establishing a medical diagnosis and provides a basis for early treatment of diseases. The permanent monitoring of their development is indispensable for measuring the effectiveness of medical treatments and modifying them if necessary, and thus for progressing toward personalized medicine.

Access to analysis of DNA sequences becomes necessary for opening up the development of “prognostics” (term referring to the identification of genetic anomalies that make the long-term development of a disease probable).

Thus, detection, for example, of a small number of copies of nucleic acid sequences in a serum, without resorting to PCR (Polymerase Chain Reaction) for their amplification, is also a major challenge for medical analysis or virology, in order to reduce the analysis times.

It is therefore essential to have measurement techniques at our disposal that are specific and very sensitive, permitting simple, rapid, routine, in situ and low-cost detection of the presence of traces of a large number of analytes (specific molecules, nano- or microparticles, bacteria, viruses, proteins, circulating biomarkers, DNA, spores etc.) dispersed in a medium which may be complex. These analytes are, for example, the 33 hazardous substances listed in the water law (Directives 76/464/EEC and 200/60/EEC), 50% of which require removal as a priority.

This framework directive on water has been an impetus for development of the sector for laboratory analysis of traces but few techniques have been proposed at the industrial level.

A study published by the French Agency for Environmental and Labor Health Safety (AFSSET) (2006) titled “Nano-materials: effects on human health and on the environment” provides a survey of the current state of techniques and limits of detection of nanoparticles in aerosols (exhaust gas, smoke) in the air, in water and in soils.

A first category comprises nonspecific detection techniques (i.e. they provide an overall characterization of all particles), which are performed without concentration of the analytes prior to analysis. These are the techniques called “Condensation Nuclei Counters” (CNC) marketed by the American company TSI and by the German company GRIMM. They consist of optically detecting the number of particles per second. Particle size can be between 5 nm and 1 μm and the concentration can be between 0.1 and 10⁶ particles/ml. They are applicable in particular in the case of air and aerosols.

The second category comprises detection techniques that are nonspecific but are performed with concentration or accumulation of the analytes prior to analysis. These are diffusion batteries in which passage of the aerosol through a succession of gratings classifies the analytes by size. Coupling to a particle counter makes it possible to determine the proportion of each granulometry of analytes with size between 2 and 200 nm. The ELPI (Electrical Low Pressure Impactor) technique selects charged particles beforehand, by inertia, with size between 7 nm and 10 μm, then detects them electrically. Their concentration must be quite high (between 1000 and 10 000 particles per ml). Electrical analysis of mobility (Scanning Mobility Particle Sizer or SMPS) makes it possible to detect particles with diameters from 3 to 50 nm with a differential mobility analyzer (DMA) and a particle counter.

Another category comprises specific detection techniques, generally proceeding with concentration prior to analysis. Atmospheric or soil particles, with size between 30 and 300 nm, are placed in solution and then analyzed, either by ion-exchange chromatography, or by mass spectrometry. Another technique uses laser-induced fluorescence (LIBS). Finally, prior fixation of nanotracers that recognize the active surface of certain nanoparticles makes it possible to detect them specifically.

We may mention, in the case of aqueous solutions of organic particles, techniques employing counting of the analytes by methods of microscopy (electron microscopy or atomic-force microscopy), by size separation (ultrafiltration, centrifugation, flow fractionation), by chromatography, by coupling fractionation with the detection technique called Inductive Coupled Plasma-Mass Spectrometry (ICP-MS), and by laser detection.

We may also mention, in the case of inorganic nanoparticles, the techniques of observation with the scanning electron microscope (SEM), diffusion of light, diffusion of X-rays or of neutrons, “Flow Field Flow Fractionation” (FFFF), hydrodynamic chromatography (HDC), electrospray and electrical mobility, zeta potential (or measurement of surface charge), atomic absorption and analysis of the light elements C—H—N—S.

However, these techniques are only suitable for analyses of size, of shape, of the state of aggregation, of surface charge and of the chemical composition of light elements. When they are specific, their limits of detection, after concentration, are poor and range from 1 g/l to 10 ng/l (or 10⁸ to 10⁹ nanoparticles/ml).

They are not readily quantitative, they are insufficiently sensitive (i.e. their limit of detection concentration is not low enough) and in particular are insufficiently specific to the particles whose detection is generally required in a complex medium. They are also too expensive, they are not portable in field conditions, they are impracticable for routine use and require highly specialized operators in laboratories.

Works relating to the detection of molecules derived from living beings (or biodiagnostics) give a much more exacting limit of detection. For example, in “Nanostructures in biodiagnostics” (N Chem. Rev. 105 (2005) 1547-1562), a first table gives the limits of detection that have been reached for nucleic acid. They fluctuate between nanomolar (nM, or 10⁻⁹ mol/l) and femtomolar (fM, or 10⁻¹⁵ mol/l), depending on the technique used. Just one method, using the products of a PCR reaction, makes it possible to reach 0.1 fM. However, this method is long because of the time required for multiplying the nucleotide chains. A second table gives the limits of detection of proteins, which also fluctuate between nM and fM. Only the barcode amplification technique makes it possible to reach 0.030 fM (30 aM) but it is complex to implement.

This examination shows that there is certainly a wide variety of techniques for detection and analysis of solutions. The “most sensitive” are developed in the laboratory, are expensive, slow, not very portable and often nonspecific. To be credible for the areas of environmental pollution and biodiagnostics, any technique for detecting traces must be of low cost, rapid, portable, selective, with sufficient resolution to reach and if possible go below the detection threshold of 10⁵ to 10⁶ nano-objects per milliliter, or if the analytes are molecules, the femto-molar threshold.

The general objective of the present invention is to provide a method of detecting and quantifying analytes present in a complex solution that is portable, rapid and selective, that can be used with many types of analytes, is economical and makes it possible to reach, or even exceed, a detection threshold of 6.10⁵ nano- or micro-objects per milliliter or, if the analytes are molecules, of the femtomolar order.

More particularly, the aim of the invention is to permit the detection of nano- or micro-particulate analytes, of specific molecular analytes and of nucleotides in a simple or complex solution. The invention also aims to permit the detection of analytes such as spores, viruses or bacteria, in a simple or complex solution. The complex solution can be conditioned beforehand so as to represent the analytes present in the air, water, soil, foodstuffs, living organisms, etc.

The present invention proposes a method of detecting target analytes of interest, present or placed in solution, combining a phase of natural concentration of the target analytes of interest in the specimen and a phase of capture of the analytes of interest by convective, directed capillary assembly on a surface provided with probes and comprising a phase of analysis of the structured surface. These probes are topographic, chemical, biological, electrostatic or magnetic units.

For this purpose, the invention relates to a method of detecting and quantifying target analytes of interest in a liquid specimen obtained from a parent solution, the liquid being able to evaporate in an atmosphere in specified conditions of evaporation, the method comprising the following steps:
b) depositing the specimen on a substrate having a micro- or nano-structured surface defining analyte capture probes, so that the liquid specimen at least partially covers the structured surface of the substrate;
c) causing controlled evaporation of the specimen in the vicinity of a liquid/substrate/atmosphere triple line, in such a way that this triple line moves, at a controlled speed, over the structured surface of the substrate, as the liquid evaporates into the atmosphere, and so that the target analytes are captured, by convective, capillary assembly directed toward the probes;
d) analyzing the structured surface of the substrate obtained after step c).

By convention, the steps of the above method are performed in alphabetical order.

To achieve high sensitivity, i.e. the possibility of detecting a very low concentration of target analytes, the invention proposes the use of a combination of an effective, reproducible technique of natural concentration of the parent solution, with specific trapping of the target analytes of interest at organized sites, called “probes”, arranged on the surface of a substrate, which permits automated analysis of the surface of the substrate.

Thus, controlled evaporation of the specimen in the vicinity of the triple line generates convection currents, which concentrate the analytes in its vicinity. Moreover, when the triple line moves, as the liquid evaporates into the atmosphere, over the structured surface of the substrate, the target analytes are driven by capillary forces toward the structured surface and then captured (immobilized) by the specific forces exerted by the probes.

According to other embodiments:
step d) can be carried out by counting the analytes captured at step c) by the micro- or nano-structured surface of the substrate, and comparing the number of target analytes captured with a conversion table, to obtain the concentration of analytes in the parent solution;
step d) can be carried out by counting the analytes captured at step c), said analytes comprising reference analytes whose concentration is known and analytes of interest whose concentration is unknown but is proportional to that of the reference analytes, and by comparing the proportions of the reference analytes and of the analytes of interest;
step b) can further comprise depositing a plate in contact with the liquid specimen, to enclose the latter between the substrate and the plate;
the plate and the substrate can be moved relative to one another in a direction of translation roughly parallel to the substrate, during controlled evaporation of the specimen;
the substrate and the specimen can be confined in an enclosure with controlled atmosphere;
during step c), it is possible to regulate the partial pressure of the components of the liquid in the controlled atmosphere;
step c) can be applied by supplying an amount of energy sufficient to cause and control the evaporation of the liquid at the triple line;
the method can comprise a step a) of pre-conditioning of the parent solution;
the pre-conditioning of the parent solution can consist of removing unwanted analytes from the parent solution, of adding new target analytes, and/or of adding solvents or new molecules promoting convective, capillary assembly on the structured surface;
the method can comprise a preliminary step of preparation of the structured surface consisting of depositing a droplet of liquid comprising probe molecules, specific to the target analytes, on the surface of the substrate, so that the droplet at least partially covers the surface of the substrate, then causing controlled evaporation of the droplet in the vicinity of a droplet/substrate/atmosphere triple line, in such a way that this triple line moves, at a controlled speed, over the structured surface of the substrate, as the liquid evaporates into the atmosphere, and so that the probe molecules attach to the surface of the substrate to create a probe network structurizing the surface of the substrate;
the method can comprise an intermediate step between step c) and step d), of fixation of fluorophores on the captured target analytes to permit counting by fluorometry during step d);
the method can further comprise, between step c) and step d), at least one step of specific differentiation of the analytes trapped at step c), by applying the substrate obtained after step c) on one or more functionalized capture surfaces;
during step c), evaporation can be controlled in such a way that the triple line moves at a constant speed;
during step c), evaporation can be controlled in such a way that the triple line moves at a variable speed;
the probes on the surface can each define a network that has an optical spectrum, step d) being performed by analyzing the optical spectrum of each network after capture of the analytes at step c); and/or
the probes on the surface can be cavities of different sizes, step c) resulting in the capture of the target analytes in micro- or nano-droplets of different sizes, trapped in the cavities of different sizes, and step d) being applied by measuring the variations in intensity of at least one physical or chemical property of each micro- or nano-droplet, by determining the volume of the “limit” micro- or nano-droplet that does not comprise any analyte, the concentration of the analyte in the parent solution being equal to 1 divided by the volume of the limit micro- or nano-droplet.

The invention also relates to an assembly for detecting and quantifying analytes for implementing the above method, comprising
a substrate having a structured surface intended for receiving a specimen of parent solution containing the analytes of interest;
a means of controlling the evaporation of the solution;
a means of analyzing the micro- or nano-structured surface of the substrate.

According to other embodiments:
the means of analysis can be able to count analytes trapped by the structured surface of the substrate, and to compare the number of analytes obtained previously with a conversion table, to obtain the concentration of analytes in the parent solution;
the detection assembly can further comprise a plate that is intended to be arranged in contact with the specimen of solution to enclose the latter between the substrate and the plate;
the substrate and the plate can be mounted movably and roughly parallel to one another in a direction of translation;
the substrate and the plate can be mounted movably relative to one another in a direction of translation, the plate being inclined relative to the substrate so as to apply the specimen of solution on the structured substrate;
the plate can be flexible;
the plate can be functionalized and/or structured;
the detection assembly can further comprise an enclosure with controlled atmosphere surrounding the substrate and the specimen;
the enclosure comprises a regulator of partial pressure of the components of the liquid in the atmosphere;
the detection assembly can further comprise channels for pumping and/or injection of a flow of gas or of gas mixture;
the detection assembly can further comprise a device for supplying thermal and/or electromagnetic energy;
the control means is coupled to at least one device for observation of the triple line for adapting the control of evaporation and adjusting the speed of displacement of the triple line to at least one desired value, over the structured surface of the substrate;
the regulator of partial pressure can be coupled to at least one device for observation of the triple line for adapting the partial pressures of the components of the liquid in the atmosphere and adjusting the speed of displacement of the triple line to at least one desired value;
the surface of the substrate can comprise structuring selected from topographic, biological, chemical, electrostatic, magnetic structurings or a combination of these structurings;
the parent solution can be a colloidal solution, a pre-conditioned solution, a pre-filtered solution, a solution that has surfactants and calibration targets, a solution incorporating targets labeled by color, by fluorescence or by an integrated barcode, or a solution incorporating target-probe couplings already effected in solutions; and/or
the parent solution and/or the specimen can comprise several types of solvents.

Other characteristics of the invention will be presented in the detailed description given below, referring to the appended drawings, which show, respectively:

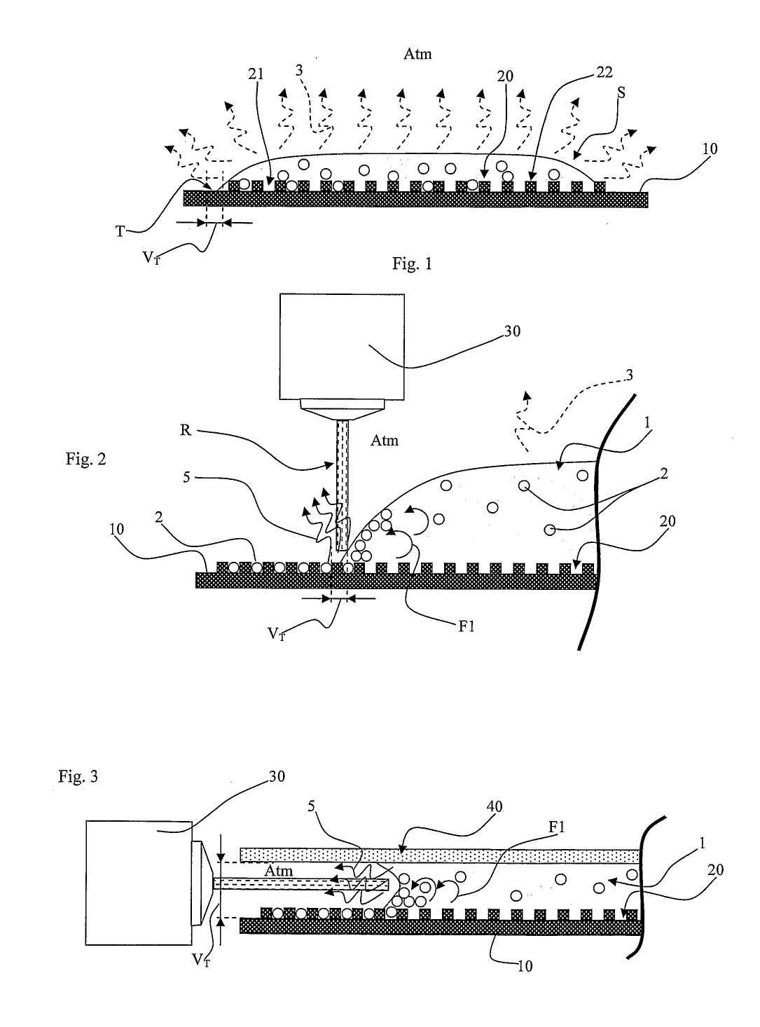

FIG. 1, a schematic profile view of a specimen deposited on a substrate and undergoing natural evaporation;

FIG. 2, a schematic profile view of a first embodiment of a device according to the invention, comprising a means of controlling evaporation in the vicinity of the triple line;

FIG. 3, a schematic profile view of a second embodiment of a device according to the invention, comprising a top plate that confines the specimen;

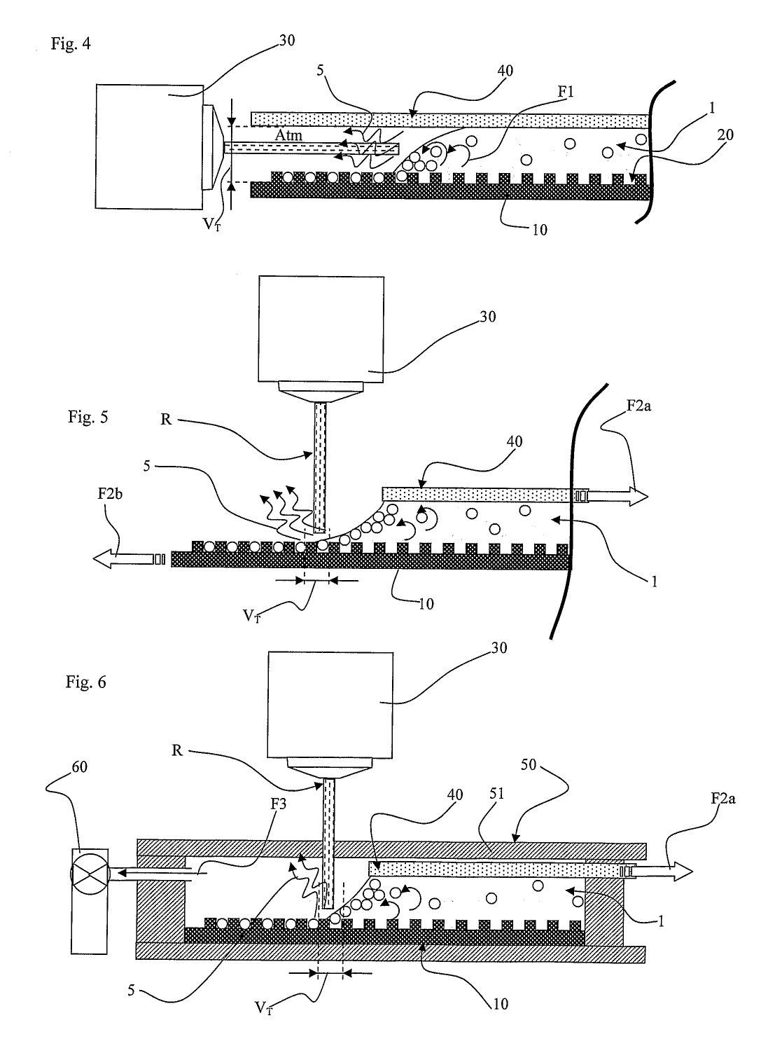

FIG. 4, a schematic profile view of a variant of the embodiment in FIG. 3, comprising a top plate that confines the specimen that is less wetting than the substrate;

FIG. 5, a schematic profile view of a third embodiment of a device according to the invention, comprising a movable top plate for confining the specimen;

FIG. 6, a schematic profile view of a fourth embodiment of a device according to the invention, comprising an enclosure with controlled atmosphere;

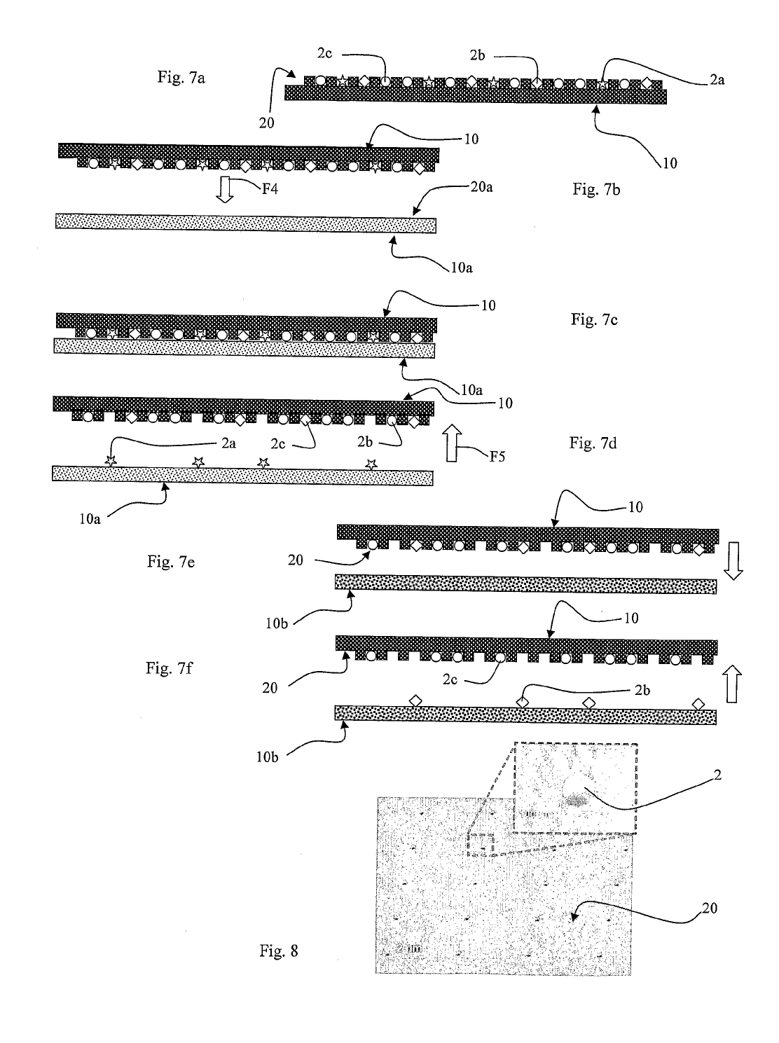

FIGS. 7a to 7f, schematic views of an advantageous embodiment of the method according to the invention, comprising two steps of specific differentiation of the analytes trapped on the surface of a substrate of a device according to the invention; and

FIG. 8, a perspective view of a substrate for detecting analytes after application of the method according to the invention.

In the following description, the analytes can be micro-objects (cells, bacteria etc.), nano-objects (nanoparticles, specific molecules (medicinal products, pesticides etc.)), biomolecules (DNA, proteins etc.), or viruses, spores, etc.

The present invention combines techniques of microelectronics and surface functionalization with techniques of convective, capillary assembly for trapping the required target analytes, on functional and specific sites, or probes, implanted and organized on a surface. This organized trapping permits a simple step of detection of the zones of probe/target coupling and therefore a simple step of analysis.

It takes place, in a general way, in three phases:

1) functionalization of a surface with predefined probe sites, densified and organized by techniques of microelectronics. These probes exert specific immobilizing forces to permit specific sampling of the targets.

2) convective, capillary assembly of a colloidal solution, optionally conditioned beforehand, containing the target analytes, which concentrates the solution naturally, on the surface of the substrate.

3) detection of the proportion of probe sites occupied by the targets, which gives, based on previous calibration, the concentration of target analytes in the solution tested.

The functionalized surface with the probe sites is represented schematically by cavities 21 in FIG. 1.

More particularly, the principle of the method according to the invention consists of sampling, i.e. taking at random, a calibrated specimen of liquid from the parent solution to be analyzed. This parent solution has an unknown concentration of nano-objects to be determined. Then the method according to the invention consists of concentrating and capturing the analytes on a structured substrate, and counting the number of analytes captured. Next, statistical analysis of one or more of these captures makes it possible to deduce, with a certain confidence interval, the concentration of the initial solution. At a fixed amount taken, the capture contains more required analytes the more the parent solution is concentrated. Moreover, the greater the number of capture sites offered, the more the analysis is representative of the composition of the parent solution.

The invention is based on controlling the evaporation of the specimen in a particular zone called the triple line. This line is the interface between the liquid of the specimen, the atmosphere in which the liquid is able to evaporate in specified conditions of evaporation (partial pressure, temperature, wettability of the substrate) and the solid substrate on which the specimen is deposited. This control makes it possible to control the displacement of the triple line over the structured surface of the substrate (see FIGS. 2 to 5).

Thus, the method according to the invention comprises a step b) of depositing the specimen on an analyte-trapping substrate, at least one portion of the surface of which is micro- or nano-structured. In this way the liquid specimen at least partially covers the structured surface of the substrate.

This surface can be structured topographically, i.e. it comprises reliefs 22 between which the analytes are immobilized. However, it can also be structured functionally. In other words, it can comprise chemical, biological, electrostatic, electrical or magnetic trapping sites. These sites are obtained, respectively, by chemical or biological probes fixed on the surface, or by the presence of electrodes supplying an electrostatic, electrical or magnetic field for trapping the analytes of interest. The surface can also be structured by zones with different wettabilities (or solubilities) relative to the solution to be tested.

Preferably, the structuring of the surface is organized according to an ordered and nonrandom pattern, to facilitate subsequent automatic analysis of the substrate.

Next, the method according to the invention comprises a step c) of controlled evaporation of the liquid (or solvent) from the specimen, roughly at the liquid/substrate/atmosphere triple line, in such a way that this triple line moves continuously over the substrate, as the liquid (or solvent) evaporates into the atmosphere.

This control localizes the evaporation of the liquid (or solvent) roughly at the triple line. In contrast, with natural evaporation, i.e. not controlled (represented by the dashed wavy arrows 3 in FIG. 1), the entire surface S of liquid in contact with the atmosphere, and not only the region of the liquid/substrate/atmosphere triple line, is subject to evaporation.

Of course, in practice, control of evaporation takes place as near as possible to the triple line, in a neighbouring zone V_{T} wider than the triple line. What matters is that the phenomenon of evaporation of the liquid (or solvent) should be predominant in the vicinity V_{T} and especially on the triple line T, relative to the rest of the surface S of the specimen 1 exposed to the atmosphere.

The method according to the invention concentrates the analytes present in the specimen naturally, and very effectively, at the triple line on account of convection currents within the liquid specimen. These convection currents appear naturally as the evaporation that is predominant starting from the triple line requires a supply of latent heat, which generates large thermal exchanges. These convection currents transport, and therefore strongly concentrate, the analytes to the vicinity V_{T} of the triple line T.

Next, the method according to the invention captures the analytes of interest, concentrated in the vicinity V_{T} of the triple line, on the substrate comprising a topographically, chemically, biologically, electrostatically, electrically or magnetically micro- or nano-structured surface.

Trapping occurs during the continuous, controlled displacement of the evaporation front (or triple line) of the solvent containing the analytes on the nano-structured surface. The capillary forces, present at the level of this triple line, direct these analytes, during the displacement of the triple line during evaporation, to precise points of the substrate (the probes) defined by the structuring. The probes select and immobilize the required target analytes, on account of specific forces. In other words, the triple line acts as the end of a natural scraper or brush which concentrates the analytes and spreads them out, plating them in the structures of surface 20 of the substrate.

The surface of the substrate is, preferably, treated so that it is predominantly nonwetting (hydrophobic if the liquid is water, solvophobic if the liquid is a solvent). This promotes confinement of the analytes by the capillary forces toward the structurings of the surface of the substrate.

The number of analyte-trapping structurings, on the surface of the substrate, determines the resolution and sensitivity of the method. As this number can be very high, the method is very sensitive. The dimensioning of the sites and/or their functionalization makes it possible to envisage analyses that are selective on the basis of shape, charge or chemical, biological or magnetic function. It is possible, for example, to create more than a million sites on 2 mm squared, which makes it possible to take a large number of analytes of the specimen on the substrate, this number being directly linked to the concentration of targets in the initial solution.

Thus, the method according to the invention performs organized capture, which facilitates automation of its execution.

This method makes it possible, during a step d), to analyze the structured surface. This analysis can be a rapid binary detection (of the type 0 or 1), and therefore a statistical measurement of the concentration in the specimen. The captures can be detected optically (reflection, phase-contrast, dark-field, fluorescence, epifluorescence, LIBS, laser beam diffraction, surface-plasmon resonance (SPR), use of nanotracers), or by electrostatic, electrical or magnetic field. Fixing of fluorescent particles on the trapped targets, and then analyzing the surface using conventional analytical scanners, can also be envisaged.

The captures of an analyte can also be located by individually structuring the probe units (for example as diffraction gratings or as clusters of di- or tri-nanoparticles) whose optical spectrum is altered by the trapping.

Thus, the probes of the micro- or nano-structured surface each define a diffraction grating that has an optical spectrum. Step d) is then executed by analyzing the optical spectrum of each diffraction grating after capture of the analytes at step c).

The concentration of analytes in the parent solution is deduced from the number of analytes detected on the substrate, using a conversion table. This table associates, for a given number of structurings on the surface of the substrate, the number of sites occupied by the analytes with the initial concentration in the parent solution.

Several variants are also possible for deducing this concentration.

For example, the concentration of an analyte in the parent solution can be obtained relatively, in relation to the known concentration of another analyte, which can be added to the parent solution, and which is detectable by its color, by reading an optical barcode defined by integrated quantum boxes, or by its fluorescence. Their proportion on the functionalized surface of the substrate gives the concentration of the required analyte directly.

The concentration of an analyte in the parent solution can also be obtained by calibrating the method of sampling in the same conditions of evaporation, from different standard solutions of identical target analytes.

The concentration of an analyte in the parent solution can also be obtained by finding, beforehand and in the same experimental conditions, the threshold concentration of analyte of a standard solution, starting from which the first filling defects of the target-receiving sites appear.

The concentration of an analyte in the parent solution can also be obtained by determining, beforehand, the experimental conditions of trapping of the targets, from standard solutions that minimize the limit concentration resulting in filling of all the sites. These conditions will be suitable for ultrasensitive analysis.

The concentration of an analyte in the parent solution can also be obtained by an intensity measurement supplied by each trap.

For this purpose, the analytes can be captured with probe units that are different (in size for example). Then several target analytes are captured per unit by trapping, for example, calibrated droplets of liquid (optionally of different volumes) on hydrophilic or solvophilic units, separated by hydrophobic or solvophobic intervals, by rapid displacement of the triple line over them. Evaporation of the solvents also creates, in the latter case, clusters of analytes.

More precisely, the micro- or nano-structured surface preferably has cavities of varying sizes. Rapid scanning of this surface by the triple line leaves micro- or nano-droplets of different volumes in these cavities of different sizes. Thus, the smaller the volume of the micro- or nano-droplet, statistically the lower the chance of finding an analyte there.

Then variations in intensity of at least one physical or chemical property of each micro- or nano-droplet are measured. For example, the variations in intensity of the optical properties of each cavity are measured after evaporation of the micro- or nano-droplets, and the curve representing the intensity as a function of the volume of the micro- or nano-droplet is plotted.

This curve makes it possible to determine the volume of the so-called “limit” micro- or nano-droplet that does not comprise any analyte. The concentration of the analyte in the parent solution is therefore equal to 1 divided by the volume of the limit micro- or nano-droplet.

The concentration of an analyte in the parent solution can finally be obtained if the yield of the different phases of trapping is known.

Preferably, the method according to the invention comprises a rinsing step prior to step c) for removing parasitic captures.

An example of calculation described below enables the performance of the method according to the invention to be evaluated.

A substrate 10 is prepared having a surface of 2 mm×2 mm with a topographic structure of dimensions suitable for trapping nanoparticles with a diameter of 100 nm. For example, surface 20 has cavities 21 with a diameter of 100 nm, with spacing of 2 μm. This substrate 10 therefore has 10⁶ probes in the form of trapping cavities 21.

A specimen is deposited on this surface in a thickness roughly equal to the diameter of the nanoparticles that it contains. The specimen comprises a known concentration of 10¹¹ target nanoparticles per ml. The diameter of each nanoparticle is 100 nm.

The volume of the specimen deposited therefore represents 4.10⁻⁷ cm³ or ml (10⁻⁵×4 10⁻²) and it carries 10¹¹×4 10⁻⁷=40 000 nanoparticles.

With natural, uncontrolled evaporation of the liquid (like that shown in FIG. 1), these nanoparticles, assumed immobile, should statistically only fill the cavities opposite which they are perfectly positioned. This sampling at random by 10⁶ cavities takes a volume of 10⁶×10⁻⁵×10⁻⁵×10⁻⁵ ml from the 4 10⁻⁷ ml of the layer, or one nanoparticle in 400, i.e. 100 nanoparticles (40 000/400) in the specimen.

With controlled evaporation according to the invention, it is observed experimentally that with an identical specimen, all the nanoparticles are trapped by the cavities of the substrate. Thus, the convection currents in the vicinity of the triple line improve the trapping of the nanoparticles. It is thus possible, with such a substrate, to trap up to 10⁶ nanoparticles if permitted by the concentration of the specimen. The method according to the invention therefore offers efficiency up to 10 000 (10⁶/100) times higher than natural, uncontrolled evaporation.

Calibration is then performed with different concentrations of parent solution. For example, the concentrations can be lower and lower. We can then draw up a conversion table between the number of sites occupied by the analytes and the initial concentration.

The method according to the invention is therefore extremely sensitive.

To attain the limit of detection of 6.10⁵ nanoparticles per milliliter (or femtomolar), it is therefore sufficient to be able to detect (10⁶/10¹¹)×(6.10⁵) clusters, i.e. 6 nano-objects captured among 10⁶ units. This number increases to 600, if a starting concentration of the specimen of 10⁹ was sufficient to fill all the units. It was thus determined experimentally that a concentration of 5.10⁸ nanoparticles per milliliter makes it possible to fill all 10⁶ topographic units. Just one capture out of 10⁶ would correspond to a concentration of analyte of less than 5×10⁸/10⁶=500 nano-objects per ml or, if the nano-objects are molecules, less than (500/6.02 10²³)×10³=8 10⁻¹⁹ mol/l=0.8 attomolar.

With 10⁸ capture sites distributed over 4 cm² the limit of detection would become 5 nano-objects per ml or 0.01 attomolar.

This calculation demonstrates that the method according to the invention makes it possible to surpass the limits of detection of the conventional techniques, simply and economically, and using very small capture surfaces, therefore with small sample volumes. It makes it possible to reach limits of detection of 500 nano-objects per ml or attomolar with 10⁶ capture sites distributed over 4 mm² and 100 times lower (5 nano-objects per ml or 0.01 attomolar) with 10⁸ structurings distributed over 4 cm².

The method according to the invention permits detection/analysis of analytes situated in a liquid environment of great complexity (water, sera, etc.) but also in the air, soil, foodstuffs, after suspending the analytes in a parent solution.

This method is simple to use, rapid, economical, portable and very sensitive. It opens up a wide range of applications extending from nano-toxicology, biodiagnostics, nano-biomedicine, pharmacology to nano-security.

Assemblies for detecting analytes, for application of the method according to the invention, are shown in FIGS. 2 to 5.

The embodiment in FIG. 2 is the simplest. A specimen 1, or droplet, comprising analytes of interest 2, is deposited on a substrate 10 with a micro- or nano-structured surface 20 (or probes) for trapping the target analytes.

Evaporation of the droplet 1 at the triple line can be promoted by creating a temperature gradient in the substrate.

Thus, this assembly can be integrated with a control means 30 of the evaporation of the specimen, arranged to cause controlled evaporation (represented by the dashed wavy arrows 5) of the specimen, roughly in the vicinity V_{T} of the triple line T. In other words, the control means promotes evaporation in the vicinity V_{T} of the triple line T which is predominant relative to the phenomenon of natural evaporation (represented by the dashed wavy arrows 3) which can occur on the rest of the surface of the specimen exposed to the atmosphere Atm.

The control means 30 can, for example, emit radiation R, suitable and calibrated for supplying an amount of energy sufficient to vaporize the liquid in the vicinity V_{T} of the triple line T. The control means can, alternatively, be a gas flow which quickly evacuates the limit layer of liquid.

As the liquid evaporates into the atmosphere, the triple line T moves over the substrate. In FIG. 2, the triple line moves from left to right.

To maintain the phenomenon of evaporation in the vicinity V_{T} of the triple line T, the control means 30 can be mounted movably in translation or on a pivot.

The control means 30 can also be coupled to at least one observation device (not shown) of the triple line T for adapting the control of evaporation by the control means 30 and/or the radiation emitted and thus regulating the speed of displacement of the triple line, to a desired value, over the structured surface of the substrate. This makes it possible to control the rate of deposition of the target analytes on the probes.

However, control of evaporation at the triple line may prove difficult in this configuration, as the specimen is in an open environment, i.e. it has a large area in contact with the atmosphere. In fact, the phenomena of natural evaporation can, depending on the atmospheric conditions, play quite an important role in reducing the phenomenon of concentration and therefore of trapping of the analytes. Moreover, evaporation takes place following the circular line of the droplet.

According to a second embodiment illustrated in FIG. 3, after depositing the specimen on the substrate, and before inducing controlled evaporation of the specimen, the method according to the invention comprises a step of depositing a plate 40 in contact with the liquid specimen 1 for enclosing the latter between the substrate 10 and the plate 40. The portion of the liquid surface that is exposed to the atmosphere in the preceding device is thus covered. The plate is preferably transparent so as to be able to observe the movement of the triple line and control the evaporation in its vicinity. For example, it can be made of glass when the specimen solvent is water.

This assembly forms a microfluidic cell which allows evaporation in a confined environment. In other words, only the vicinity V_{T} of the triple line T is exposed to the atmosphere Atm. This assembly offers better control of evaporation, leading to greater reproducibility of filling of the probe capture sites.

The control means 30 causes evaporation which forms a meniscus in specimen 1, between the plate 40 and the substrate 10, and in the vicinity V_{T} of the triple line T. The backward movement of this meniscus, toward the right in FIG. 3, as evaporation proceeds, causes a displacement of the triple line T, also toward the right in FIG. 3. In this assembly, there is also a triple line between the specimen, the atmosphere and the plate 40. However, this plate 40 is not structured, so that the analytes do not become attached to the plate 40. It can also be structured to prevent the analytes attaching to it. Owing to the convection currents F1, the analytes are concentrated toward the structured surface of the substrate 10.

The substrate 10 is treated so that it is only partially wetting and assembles the analytes toward the units using capillary forces. The units select the targets by fixing them under the action of their specific forces.

In the embodiment depicted in FIG. 4, plate 40 has received a surface treatment which makes it less wetting than the substrate. It then pulls the triple line, toward the right in FIG. 4, as evaporation proceeds, and directs the displacement of the triple line over the substrate 10. Assembly is quasi-static.

This equilibrium between the wettability of the substrate and that of the top plate is delicate. The substrate must not be too hydrophilic to avoid compact assembly by pure convection between the structurings. In case of difficulties, a double functionalization of the substrate (attraction in the units and repulsion outside of the units) will be an effective variant. Another solution is to structure the plate.

A variant of the preceding microfluidic cell, illustrated in FIG. 5, adds controlled translation of the top plate 40 in the direction of the arrow F2a. The direction of translation (arrow F2a) is roughly parallel to the plane of the substrate 10. This translation offers the advantage of controlling the spreading of the meniscus near the triple line and therefore trapping of the analytes in the structures of the surface 20. Alternatively or in combination, the controlled translation can be that of the substrate 10, in the direction of the arrow F2b. What is important, in this embodiment, is that a relative movement is applied between the substrate 10 and the plate 40.

It is also possible to provide a device for adjusting the distance between the top plate 40 and the substrate 10 in order to adjust the height of the meniscus.

The top plate 40 can be slightly inclined and can be made of a flexible material and can be displaced, in a direction of translation F2a, so as to perform the role of a scraper (or of a brush) that applies a colloidal solution on the structured substrate.

The inclination of plate 40 can be adjustable.

The detection assemblies shown in FIGS. 2 to 5 can be placed in an enclosure with controlled atmosphere, which is isothermal or has a temperature gradient, so as to control the speed of movement of the triple line.

One embodiment, illustrated in FIG. 6, combines the microfluidic cell with movable top plate 40 (and/or with movable substrate 10), illustrated in FIG. 5, with an enclosure 50 surrounding the substrate 10, plate 40 and specimen 1. The enclosure 50 makes it possible to control the atmosphere, and not only to confine it, as with the microfluidic cell alone.

In a simpler embodiment (not shown), the top plate is not movable. It may then be unnecessary if the enclosure provides the same function by comprising a cover 51 that can come into contact with the specimen 1.

The enclosure is preferably combined with a regulator 60 of partial pressure of the components of the liquid of the specimen (solvents and solutes) in the atmosphere.

For example, if the solvent of the specimen is water, when the specimen evaporates in the vicinity of the triple line, the water partial pressure in the atmosphere increases. The kinetics of the phase transition is therefore altered, as it becomes more difficult for the solvent to evaporate.

The regulator 60 can keep the partial pressure below the threshold value of saturated vapor pressure of water. It then actuates evacuation of some of the water in the form of vapor (according to arrow F3) so that the specimen can continue to evaporate. The triple line T then moves in a controlled manner, owing to this evacuation, over the surface 20 of the substrate 10.

The regulator 60 can also keep the partial pressure above the aforementioned threshold value. It then blocks the evacuation of water in the form of vapor, which stops the evaporation of the specimen. The choice of water partial pressure in the enclosure therefore regulates the speed of movement of the triple line T over the surface 20 of the substrate 10. In the case of a solution comprising several solvents it is possible, by this mechanism, to block the evaporation of one of them.

The regulator 60 can also be coupled to an observation device (not shown) of the triple line T for adjusting the speed of movement of the triple line to the desired value over the structured surface of the substrate, by the choice of partial pressure.

Thus, by regulating the partial pressure of the atmosphere, it is possible to act on the speed of movement of the triple line over the substrate. In doing so, the trapping of the analytes by the structured surface 20 of the substrate 10 is optimized.

The regulator 60 can also control the temperature of the atmosphere or create a gradient on the substrate.

A device for sucking or blowing gas, integrated with the plate, can also control the speed of movement of the triple line by faster evacuation of the limit layer of evaporation (evacuation of the vaporized molecules of solvent).

It is thus possible to force, by capillarity and the action of specific forces, the capture of micro-objects (bacterial cells etc.), of nano-objects (nanoparticles, specific molecules such as medicinal products or pesticides), of biomolecules (DNA, proteins etc.) or of viruses, spores, etc.

The method according to the invention can comprise, prior to step c) of analysis of the surface, one or more additional step(s) of specific differentiation of the trapped analytes, by applying the substrate obtained after step b) on one or more pre-functionalized surfaces. It is then possible for specific target analytes of the pre-functionalized surface used to be extracted from the substrate. Then the surfaces thus obtained are analyzed according to the aforementioned step c). This step of specific differentiation of the trapped analytes is shown in FIGS. 7a to 7f.

FIG. 7a shows a substrate 10 obtained after step b). Three types of analytes 2a, 2b and 2c have been captured by the functionalized surface 20 of the substrate 10.

The surface 20 of the substrate 10 is applied, in the direction of the arrow F4, against a substrate 10a, provided with a functionalized surface 20a capable of specifically fixing the analytes 2a (FIGS. 7b and 7c).

Then the substrate 10 is withdrawn from the substrate 10a, in the direction of the arrow F5. The substrate 10a therefore withdraws the analytes 2a from the surface 20 of the substrate 10 (FIG. 7d).

The operation is repeated with a substrate 10b, provided with a functionalized surface 20b capable of specifically fixing the analytes 2b.

The surface 20 of the substrate 10, obtained after the step of specific differentiation shown in FIGS. 7b to 7d, is applied against substrate 10b (FIG. 7e).

Then the substrate 10 is withdrawn from the substrate 10b in the direction of the arrow F5. The substrate 10b therefore withdraws the analytes 2b from the surface 20 of the substrate 10 which now only comprises analytes 2c.

Finally, the surface of the substrates 10, 10a and 10b thus obtained is analyzed according to the aforementioned step c).

The functionalization of the surfaces 20a-20b must be adapted so that the force of transfer, i.e. of attraction of the surface 20a-20b is greater than that of retention of the analytes 2a-2b in the units of the surface 20 of the substrate 10. Advantageously, for substrate 10, substrates are used that have lower surface energy, such as PDMS, than the surfaces 20a and 20b.

These additional step(s) of specific differentiation of the trapped analytes permit automatic, specific analysis of each type of analytes.

FIG. 8 shows the structured surface of the analyte detection substrate after carrying out the method according to the invention. In this diagram, nanoparticles 2, with a diameter of 100 nm, are trapped in cavities with spacing of 2 μm on the structured surface 20 of a substrate 10 and then transferred onto a glass substrate 10a.

The method according to the invention can be applied in many branches of industry. More particularly:
nano-toxicology (i.e. detection of nano-objects generated artificially by humans and dispersed in the environment): detection of specific molecules (pesticides, medicinal products, detergents etc.), of nanoparticles or of combustion products (ash, dioxins etc.); optionally, it allows their harmful effects to be studied.
nano-biomedicine, i.e. medical analysis, development of micro- and nano-techniques suitable for earlier and earlier detection of biological abnormalities (DNA, proteins, etc.), detection of viruses or bacteria, the use of nanoparticles as an analysis “vector”, investigation of media that are favorable or unfavorable to the multiplication of viruses and of bacteria, etc.
pharmacology for screening medicinal products.
nano-security by ultrasensitive detection of spores, viruses, bacteria, etc.
treatment of surfaces and in particular healing of their surface defects.

According to other embodiments:
The functionalization of the surface of the substrate can comprise:
Overall functionalization of the very hydrophilic surface, called “convective assembly”, to obtain compact assembly of the analytes on the surface;
The manufacture of topographic units of different diameters (for sorting the nano-objects by size), of different heights (to permit recovery of the targets of interest by buffering on another substrate), of different spacings and arrangement geometries (for better densification and/or to exploit total reading techniques);
The manufacture of the probe units by chemical contrasts promoting interactions with targets of the covalent bond type (thiol type) and/or hydrogen bonds and/or by van der Waals forces (carbon-containing radical), amine bonding, ionic bonding by dipole/dipole interaction, and/or not promoting these interactions in the spaces between the units (by chemical contrasts such as octadecyl trimethoxysilane (OTS), aminopropyl trimethoxysilane (APTES), etc., by hydrophilic/hydrophobic—solvophilic/solvophobic contrasts);
The manufacture of probe units by biological contrasts such as biotin, streptavidin, polyethylene glycol, etc.;
The use of passivating surface chemistry (such as OTS, PEG—Poly Ethylene Glycol—or BSA—Bovine Serum Albumin—, etc.) of certain zones to forbid the trapping of antibodies, of peptides and of DNA between the units;
The localized injection of positive or negative charges to trap polarized or polarizable analytes;
The use of magnetic traps such as spherical nanoparticles, rods, tapes, tori and/or stacks thereof;
The combination of two or more of the aforementioned procedures;
The use of target substrates in different materials such as glass, silicon, silicon oxide, PDMS (polydimethylsiloxane), ITO (Indium Tin Oxide) with high or low surface energy whose optical properties such as reflectivity prepare the analysis;
The use of substrates having a surface area different from 4 mm² and having a number of trapping sites different from 10⁶.
The parent solution can be selected from:
natural colloidal solutions of targets;
water-based solutions;
solutions based on solvents of different nature: organic, ether, acetone, chloroform, octane, heptane, nonane, decane, trichloroethylene;
specimens of sera, of blood, of biological organs;
solutions of solids in a suitable solvent;
recovery and pre-concentration, by accumulation in a suitable solvent, of analytes present in the air, in aerosols or in any complex medium,
solutions in which the required target/probe trapping has already been carried out, wherein the probes can be supported (pre-substrated) by nanoparticles with integrated barcode (defined by quantum boxes) defined by color, fluorescence, etc.;
targets labeled with fluorescent markers;
a complex solution.
solutions conditioned beforehand by filtering, by removal of unnecessary targets.
The introduction of surfactants in the parent solution (Triton X, etc.) and of solvents (which is also a pre-conditioning) to facilitate assembly;
The introduction, in the parent solution, of a calibration target of known concentration and behavior so that relative concentrations can be determined.
The deposition of the specimen of parent solution on the substrate can exploit:
either the wetting character (hydrophilic for water, solvophilic for a solvent) of the substrate in order to spread out the droplet deposited, or conversely an intermediate wetting/dewetting character (partially hydrophobic for water) to limit its spreading beyond the structured zone on the substrate.
Depositing a top plate on the droplet with the aim of defining a layer of liquid of controlled thickness and of preventing evaporation of the solvent other than at the triple line.
This top plate can be functionalized to be nonwetting (more hydrophobic for water than the substrate) with the aim of forcing (pulling) the displacement of the triple line naturally;
This plate can be slightly inclined to define a quasi-rectilinear triple line of evaporation, to form a well-defined meniscus at its end and minimize the surface of contact with the air.
This top plate can be structured
Step b) of controlled evaporation can exploit either:
natural evaporation that is more effective at the triple line than in the rest of the volume of the droplet, and which concentrates the analytes on this line, on account of convection currents; this natural evaporation can be controlled by applying a temperature gradient between the triple line and the rest of the droplet;
forced evaporation of the solvent in the vicinity of this triple line by a local reduction in partial pressure of the solvent (pumping), faster evacuation of the evaporated limit layer (by a gas flow) or aspiration, heating, laser illumination, heating of the corresponding portion of the substrate creating a temperature gradient on the substrate, etc;
depositing a plate in contact with the droplet of liquid, which encloses the latter between the substrate and the plate, which limits and confines the evaporation in the region near the triple line;
Step b) of controlled evaporation can be used for:
first assembling the probes, then repeated for assembling the targets on these probes;
taking micro- or nano-droplets of solution by rapid displacement of the triple line, thus trapping arrangements of analytes or performing micro-nano sampling or micro-nano laboratories
a device for tracking the displacement of the triple line can be integrated for automating the deposition of the liquid specimen on the substrate, based on feedback electronics coupled to image analysis;
the substrate can be of a first material and can be covered, completely or partially, with structured capture layers of different materials.
the analyte detection assembly can further comprise channels for pumping and/or injection of a gas flow. In particular, in the embodiments shown in FIGS. 3 to 6, the plate 40 can support these channels for pumping and/or injection of a gas flow;
the parent solution can be a colloidal solution, a solution incorporating surfactants and calibration targets, a solution incorporating fluorescence-labeled targets or a solution incorporating target-probe couplings already effected in solution. In the latter case, the structured surface of the substrate 10 can be selected for fixing the probe, the target or the assembly preferentially or successively;
the parent solution and/or the specimen can comprise several solvents.

One of the novel features of this invention is controlling the evaporation of a solvent in the vicinity of the triple line which concentrates the required target analytes naturally on this line in a compact arrangement. This phenomenon of concentration arises from the generation of convection currents in the evaporating liquid, whose role is to supply the thermal energy (or latent heat) required for evaporation. The use of controlled evaporation of the specimen, deposited on a structured surface, permits analyses by size or by chemical, biological, electrostatic, electrical and/or magnetic properties of the analytes.

Another novel feature consists of reaching a very low limit of detection by effecting sampling on a large number of functional or selective receiving probe sites occupying a very small surface area. The invention is therefore suitable for analysis of specimens of a few hundredths of a microliter.

Moreover, the organization of the probes makes it possible to use automated techniques for detecting probe-target capture. The invention thus makes it possible to obtain a well-defined pattern of sites, occupied by the required analytes and unoccupied, and analysis that is simple and can be automated.

The invention therefore proposes a laboratory on a chip, which speeds up and lowers the costs of the analyses, and permits numerous analyses in parallel.

1. A method of detecting and quantifying target analytes of interest in a specimen of liquid obtained from a parent solution, the liquid being able to evaporate in an atmosphere in specified conditions of evaporation, the method comprising the following steps:
b) depositing the specimen on a substrate having a micro- or nano-structured surface defining analyte capture probes, so that the liquid specimen at least partially covers the structured surface of the substrate;
c) causing controlled evaporation of the specimen in the vicinity of a liquid/substrate/atmosphere triple line, constituted by the interface between the liquid of the specimen, the atmosphere and the substrate, in such a way that this triple line moves, at a controlled speed, over the structured surface of the substrate, as the liquid evaporates into the atmosphere, and so that the target analytes are captured, by convective, capillary assembly directed toward the probes;
d) analyzing the structured surface of the substrate obtained after step c).

2. The method of detecting and quantifying analytes as claimed in claim 1, in which step d) is applied by counting the analytes captured at step c) by the micro- or nano-structured surface of the substrate, and by comparing the number of target analytes captured against a conversion table, to obtain the concentration of analytes in the parent solution.

3. The method of detecting and quantifying analytes as claimed in claim 1, in which step d) is applied by counting the analytes captured at step c), said analytes comprising reference analytes whose concentration is known and analytes of interest whose concentration is unknown but proportional to that of the reference analytes, and by comparing the proportions of the reference analytes and of the analytes of interest.

4. The method of detecting and quantifying analytes as claimed in claim 1, in which step b) further comprises depositing a plate (40) in contact with the liquid specimen (1), for enclosing the latter between the substrate (10) and the plate (40).

5. The method of detecting and quantifying analytes as claimed in claim 4, in which the plate and the substrate are displaced relative to one another in a direction of translation roughly parallel to the substrate, during controlled evaporation of the specimen.

6. The method of detecting and quantifying analytes as claimed in claim 1, in which the substrate and the specimen are confined in an enclosure with controlled atmosphere.

7. The method of detecting and quantifying analytes as claimed in claim 6, in which the partial pressure of the components of the liquid in the controlled atmosphere is regulated during step c).

8. The method of detecting and quantifying analytes as claimed in claim 1, in which step c) is applied by supplying an amount of energy sufficient to cause and control the evaporation of the liquid at the triple line.

9. The method of detecting and quantifying analytes as claimed in claim 1, comprising a step a) of pre-conditioning of the parent solution.

10. The method of detecting and quantifying analytes as claimed in claim 9, in which the pre-conditioning of the parent solution consists of removing unwanted analytes from the parent solution, of adding new target analytes, and/or of adding solvents or new molecules promoting convective, capillary assembly on the structured surface.

11. The method of detecting and quantifying analytes as claimed in claim 1, comprising a preliminary step of preparation of the structured surface consisting of depositing a droplet of liquid comprising probe molecules, specific to the target analytes, on the surface of the substrate, so that the droplet at least partially covers the surface of the substrate, then causing controlled evaporation of the droplet in the vicinity of a droplet/substrate/atmosphere triple line, in such a way that this triple line moves, at a controlled speed, over the structured surface of the substrate, as the liquid evaporates into the atmosphere, and so that the probe molecules attach to the surface of the substrate to create a probe network structuring the surface of the substrate.

12. The method of detecting and quantifying analytes as claimed in claim 1, comprising an intermediate step between step c) and step d), of fixation of fluorophores on the captured target analytes to permit counting by fluorometry during step d).

13. The method of detection and quantification as claimed in claim 1, further comprising, between step c) and step d), at least one step of specific differentiation of the analytes trapped at step c), by applying the substrate obtained after step c) on one or more functionalized capture surfaces.

14. The method of detection and quantification as claimed in claim 1, in which, during step c), evaporation is controlled in such a way that the triple line moves at a constant speed.

15. The method of detection and quantification as claimed in claim 1, in which, during step c), evaporation is controlled in such a way that the triple line moves at a variable speed.

16. The method of detection and quantification as claimed in claim 1, in which the probes of the surface each define a grating providing an optical spectrum, step d) being performed by analyzing the optical spectrum of each grating after capture of the analytes at step c).

17. The method of detection and quantification as claimed in claim 1, in which the probes of the surface are cavities of different sizes, step c) resulting in capture of the target analytes in micro- or nano-droplets of different sizes, trapped in cavities of different sizes, and step d) being applied by measuring variations in intensity of at least one physical or chemical property of each micro- or nano-droplet, by determining the volume of the “limit” micro- or nano-droplet which does not contain any analyte, the concentration of the analyte in the parent solution being equal to 1 divided by the volume of the limit micro- or nano-droplet.

18. An assembly for detecting and quantifying analytes for implementing the method as claimed in claim 1, comprising
a substrate having a structured surface intended for receiving a specimen of parent solution containing the analytes of interest;
a means of control of the evaporation of the solution in the vicinity of a liquid/substrate/atmosphere triple line;
a means of analyzing the micro- or nano-structured surface of the substrate.

19. The assembly for detecting and quantifying analytes as claimed in claim 18, in which the means of analysis is able to count analytes trapped by the structured surface of the substrate, and to compare the number of analytes obtained previously against a conversion table, to obtain the concentration of analytes in the parent solution.

20. The assembly for detecting and quantifying analytes as claimed in claim 18, further comprising a plate intended to be arranged in contact with the specimen of solution to enclose the latter between the substrate and the plate.

21. The assembly for detecting and quantifying analytes as claimed in claim 20, in which the substrate and the plate are mounted movably and roughly parallel to one another in a direction of translation.

22. The assembly for detecting and quantifying analytes as claimed in claim 21, in which the substrate and the plate are mounted movably relative to one another in a direction of translation, the plate being inclined relative to the substrate so as to apply the specimen of solution on the structured substrate.

23. The assembly for detecting and quantifying analytes as claimed in claim 22, in which the plate is flexible.

24. The assembly for detecting and quantifying analytes as claimed in claim 20, in which the plate is functionalized and/or structured.

25. The assembly for detecting and quantifying analytes as claimed in claim 18, further comprising an enclosure with controlled atmosphere surrounding the substrate and the specimen.

26. The assembly for detecting and quantifying analytes as claimed in claim 25, in which the enclosure comprises a regulator of partial pressure of the components of the liquid in the atmosphere.

27. The assembly for detecting and quantifying analytes as claimed in claim 25, further comprising channels for pumping and/or injecting a flow of gas or of gas mixture.

28. The assembly for detecting and quantifying analytes as claimed in claim 18, further comprising a device for supplying thermal and/or electromagnetic energy.

29. The assembly for detecting and quantifying analytes as claimed in claim 18, in which the control means is coupled to at least one device for observation of the triple line for adapting the control of evaporation and adjusting the speed of displacement of the triple line to at least one desired value, on the structured surface of the substrate.

30. The assembly for detecting and quantifying analytes as claimed in claim 26, in which the regulator of partial pressure is coupled to at least one device for observation of the triple line for adapting the partial pressures of the components of the liquid in the atmosphere and for adjusting the speed of displacement of the triple line to at least one desired value.

31. The assembly for detecting and quantifying analytes as claimed in claim 18 any one of claims 18 to 30, in which the surface of the substrate comprises a structuring selected from topographic, biological, chemical, electrostatic, magnetic structurings or a combination of these structurings.

32. The assembly for detecting and quantifying analytes as claimed in claim 18, in which the parent solution is a colloidal solution, a pre-conditioned solution, a pre-filtered solution, a solution that has surfactants and calibration targets, a solution incorporating targets labeled by color, by fluorescence or by an integrated barcode, or a solution incorporating target-probe couplings already effected in solutions.

33. The assembly for detecting and quantifying analytes as claimed in claim 18, in which the parent solution and/or the specimen comprises/comprise several types of solvents.
